(19) United States
(12) Patent Application Publication
(10) Pub. No.: US 2006/0270021 A1
(43) Pub. Date: Nov. 30, 2006

(54) Integrated microfluidic sperm isolation and insemination device

(76) Inventors: Shuichi Takayama, Ann Arbor, MI (US); Gary D. Smith, Ann Arbor, MI (US); Ronald Suh, Ann Arbor, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 10/559,742

(22) PCT Filed: Jun. 7, 2004
(86) PCT No.: PCT/US04/17829

§ 371 Date: May 24, 2006

(51) Int. Cl. C12M 1/00

(52) U.S. Cl. 435/283.1

(57) ABSTRACT

An integrated microfluidic sperm isolation and oocyte insemination device provides the opportunity to perform in vitro insemination with motilityenhanced sperm samples and with minimal manipulation of fragile oocytes. Sperm sorting is performed in a common sort channel wherein more mobile sperm swim across the interface between co-laminar flows of semen and media fluid.

### CROSS-REFERENCE TO RELATED APPLICATIONS

This application claims the benefit of U.S. provisional application Ser. No. 60/476,664 filed Jun. 6, 2003.

### BACKGROUND OF THE INVENTION

1. Field of the Invention

The present invention is directed to an integral microfluidics device which sorts sperm based on sperm motility and utilizes sorted sperm for oocyte insemination, to a method for its use, and to a further method for microfluidic insemination of oocytes.

2. Background Art

Conventional in vitro insemination techniques involve separation of more motile sperm from non-motile sperm, deformed sperm, cellular debris, etc.; by techniques which can damage the sperm. Even so, hand sorting is frequently necessary. Use of the sperm to fertilize an oocyte also generally involves numerous manipulative steps which can cause damage to the fragile oocytes. For example, intracytoplasmic sperm injection has been used, particularly when the availability of viable sperm is very low, as but a single sperm is injected into the egg by micromanipulation techniques. However, up to 5-7% of oocytes may lyse during this process. Insemination in standard center-well culture dishes is better in this respect, but fertilization rate drops considerably when the sperm concentration is low.

Artificial insemination is frequently practiced in humans when married couples experience fertility problems. However, artificial insemination may also be desired in animal species to enable some control on the gender of the offspring, or for other reasons. Since these procedures involve complex, microscopic manipulative steps, as described earlier, they are, in general, quite expensive.

It would be desirable to provide a device suitable for in vitro insemination which is self contained, inexpensive, and which allows for both sperm sorting and oocyte insemination while minimizing manipulative steps, thus decreasing cost and increasing fertilization efficiency. It would be further desirable to provide an insemination method which is more effective at low sperm concentration than traditional culture methods.

### SUMMARY OF THE INVENTION

An integral device which both sorts sperm based on motility and then uses the sorted sperm to inseminate an oocyte, consists of minimally two gravity driven pumps, one for semen and one for a media fluid, and a common sort channel wherein more motile sperm swim across the interface between co-laminar flows of semen and media fluid. The media fluid, now enriched with more motile sperm, is used “on chip” to fertilize trapped oocytes, or may be used with a simple and separate microfluidic device to fertilize trapped oocytes.

### BRIEF DESCRIPTION OF THE DRAWINGS

FIG. 1 illustrates schematically the sperm sorting portion of one aspect of the invention.

FIGS. 2a, 2b, and 2c illustrate the principle of sperm sorting relied upon in one aspect of the present invention.

FIG. 3 illustrates one embodiment of a device of one aspect of the subject invention.

FIG. 4 illustrates one embodiment of a barrier utilized to trap oocytes in an insemination chamber.

FIG. 5 illustrates a simple Gravity Driven Horizontal Pump.

FIG. 6 illustrates the surprisingly increased efficiency of microfluidic insemination at low sperm levels.

FIG. 7 illustrates a further embodiment of the invention.

### DETAILED DESCRIPTION OF THE PREFERRED EMBODIMENT(S)

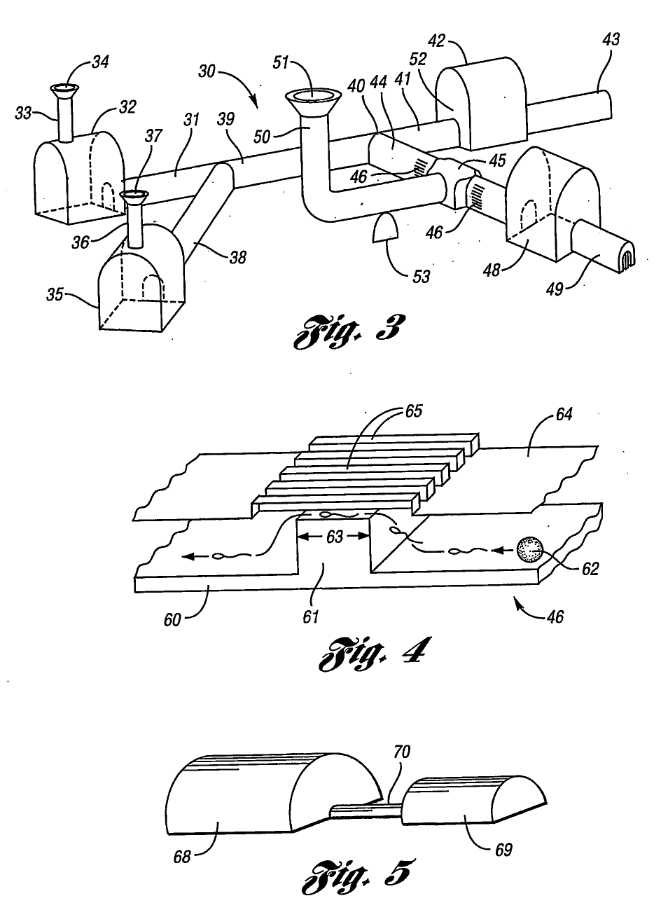

The integral devices of the subject invention sorts sperm “on chip”, and rely on the ability of motile sperm to cross the interface between co-laminar flow of a sperm-containing liquid such as semen, and a media fluid in which the sperm remains viable. The principles involved are illustrated in FIG. 2, in which streams of media fluid and semen converge in a common sort channel, and sperm of higher motility cross the interface between the co-flowing streams. The streams are then separated. The result is a media fluid enriched with sperm of higher motility. The process may be repeated in parallel or in series, or both, to create highly efficient sperm sorting. Such sorting is described in Schuster, et al., “Isolation of Motile Sperm From Semen Samples Using Microfluidics”, REPROD. BIOMED. ONLINE 7, 75-81.

In order to provide efficient sorting, laminar flow is necessary. With microfluidics devices, the small volume channels require very little liquid, and providing a constant delivery is difficult with traditional syringes, pumps, etc. Fluctuations in volume or pressure can cause disturbances which may cause the fluid flow to become turbulent. This turbulence causes a mixing effect which can destroy the ability of the sort channel to sort sperm. To provide a constant, low volume flow, the subject invention devices incorporate a gravity driven horizontal pump with a constant head. The constant head facilitates an exceptionally uniform flow rate. The gravity driven pump, like the sperm sorting portion of the inventive device will be described later in greater detail.

The final component of the subject invention integral device is an insemination chamber. Sorted sperm enter the insemination chamber either directly from the sort channel, or following initial accumulation in a sorted sperm reservoir. One or more oocytes are first deposited in the chamber and are prevented from exiting the chamber by a suitable barrier which allows fluid and sperm to flow, but prevents the oocyte from doing so. The insemination chamber may also be used in and of itself, with sperm which has been isolated by laminar flow sperm sorting or conventional sperm sorting. It has been surprisingly discovered that at low sperm concentrations, microfluidic insemination is more efficient than conventional culture techniques such as center-well, culture tubes, or microdrops under oil.

Due to the very small forces involved in all phases of sperm sorting and oocyte insemination as disclosed herein, the risk of failure of insemination, as well as damage to sperm, oocyte, or zygote are minimized. The devices may be prepared from numerous materials. However, due to its substantially biologically inert character and the ease of its use, castable silicones are preferably employed. The device will now be described in greater detail, in terms of its components.

The gravity driven microfluidic pump system comprises horizontally-oriented fluid supply reservoir(s) and a horizontally oriented outlet reservoir(s) connected via microchannel(s) in which the pump fluid flows. The fluid supply reservoirs are substantially horizontal, and are of sufficient size to carry the desired amount of fluid for sustained operation. By the term “substantially horizontal” is meant a horizontal or nearly horizontal position such that the hydrostatic pressure asserted by the liquid in the supply reservoir is relatively constant. A horizontal reservoir may deviate from the absolute horizontal orientation to generate a pumping system where the hydrostatic pressure is intentionally caused to vary over time. In general, an orientation which deviates from horizontal by about 100 or less, preferably 5° or less is suitable. A very slight incline toward the outlet of the supply, i.e. 1-3°, may be useful to counter effects due to surface tension between the liquid being pumped and the walls of the reservoir. Due to the volume generally required to supply fluid over time, the volume of the supply reservoir is normally considerably greater than the volume of the microchannel(s) through which fluid flow is desired. For example, at flow rates of 1000-3000 nL/hr, the reservoir may be several mL in volume. Gravity driven pumps deliver liquid at an essentially constant “head.”

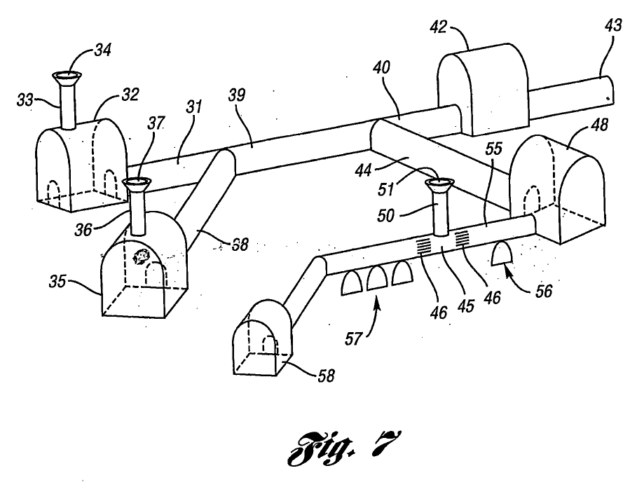

The reservoir may constitute a straight run of hollow section and may also be bent in a U-configuration or in a horizontal spiral to conserve space. A suitable configuration is shown in FIG. 5, which will later be described in greater detail. The internal cross-section of the hollow section may be of any desired shape. Preferably, the internal cross-section is half-round, but elliptical, square, rectangular, octagonal, (“polyhedral”) or other cross-sections may be used as well, as long as the shape is such that it maintains enough surface tension to keep the fluid inside from spilling out. The liquid spans the entire height of the reservoir, even when nearly empty, due to surface tension. The cross-sectional shape may vary along the length of the reservoir to program different hydrostatic pressures at different points in time determined by the amount of fluid volumes present in the reservoirs. A separate supply reservoir may be connected to the rnicrofluidic device to supply fluid for more extended periods. For example, a glass tubing reservoir may have standard microfluidics connectors which enable connection to a microfluidic device channel through suitable tubing, for example hollow tubing of polydimethylsiloxane elastomer (“PDMS”), polysulfone, etc. In such devices, at least a portion of the supply reservoir and the microchannel(s) are at different heights, so that when positioned such that the supply reservoir is horizontal, a gravity-created hydrostatic pressure condition exists between the reservoir and the microchannel outlet. The microchannel may be horizontal, inclined or vertical, preferably horizontal.

The “diameter” or cross-sectional dimensions of the fluid supply reservoir are only critical to the extent that the surface tension between the fluid and the walls of the reservoir is sufficient to maintain the liquid within the reservoir at a given reservoir internal diameter, and the internal diameter is not so small as to prevent fluid flow due to the surface tension. This relationship between surface tension and internal diameter will vary depending upon several factors, including the geometry of the internal cross-section of the reservoir, the nature of the inner walls of the reservoir, and the nature of the fluid. The suitability of any particular reservoir may be assessed easily by filling the reservoir with liquid and observing whether the reservoir will hold the liquid stably while pumping liquid through the microchannel when connected thereto.

For example, reservoirs of square or rectangular cross-section will be expected to allow for larger “diameters” than those of circular cross-section due to increased interaction between the fluid and the walls at the corners of the internal cross-section and the higher surface to volume ratio of non-circular cross-sections. For aqueous fluids, internal walls which are hydrophilic will exhibit lesser fluid/wall interactions than surfaces which are less hydrophilic (or more hydrophobic). Finally, the nature of the fluid itself is important. The presence of surface tension-lowering compounds in the fluid will alter the maximum “diameter” accordingly.

For example, with a fluid which contains 1.0 weight percent BSA (bovine serum albumin), it was found that 5-6 mm inside diameter (“I.D.”) reservoirs are incapable of holding fluid by surface tension, whereas a reservoir of 2.5 mm exerted high surface tension. However, reservoirs with nominal diameters between 3.5 mm and 4.0 mm were found to be eminently well suited as a reservoir for such applications. The suitability for any given internal “diameter”/configuration can be simply assessed by ascertaining, first, whether fluid can be held within the reservoir by orienting the reservoir horizontally and determining that the fluid will not flow out of its own accord; and second, by ascertaining that the interaction between the wall of the reservoir and the fluid is not so high that the desired flow rate between the reservoir and a given microchannel cannot be obtained. Channels in the microfluidic device exhibit fluid flow due to hydrostatic pressure created by a difference in height (relative to gravitational field) between the supply reservoir and the microchannel.

The sort channel of the device is terminated in an outlet. The outlet is preferably in fluid communication with an outlet fluid reservoir, in order to prevent fluctuations in pressure associated with formation of “drops” from the outlet. The outlet reservoir may be a simple container with which the microchannel communicates, or preferably is an outlet reservoir of the same size, material, and geometry as the supply reservoir, but with a lower height and/or vertical position. In such a situation, capillary forces substantially cancel out and hydrostatic pressure due to gravity is the only driving force for the liquid.

When the supply reservoir and outlet reservoir are of the same configuration and construction, the effects of geometry-induced and constitution-induced (i.e. hydrophilic/hydrophobic) properties may be completely offset, thus increasing the constancy of flow. In such cases, once the device is initially filled with fluid, i.e. by application of a slight pressure differential between the supply reservoir and the outlet, fluid flow may occur from reservoirs whose internal size is too small for flow without the use of a similar outlet reservoir.

The integral devices of the subject invention contain minimally two fluid supply reservoirs, one for the sperm to be sorted, and one for the fluid media which will receive higher motility sperm. In addition the integral devices may contain further reservoir(s) for supplying fluid streams of different composition, e.g. different nutrients, growth factors, etc., or to present a larger supply of liquid than is possible in a single reservoir alone. It should be remembered that although the length of fluid supply reservoirs can be increased easily, their cross-section cannot, since too large a cross-section will prevent the fluid from occupying the entire height of the reservoir. These considerations do not necessarily apply to the outlet reservoirs, although these also preferably are constructed with regard to the same criteria.

The passageways of the subject devices, for example those from or to the various fluid reservoirs, may be valved. Valving is particularly important when multiple media reservoirs are employed. Valving may be by active microvalves embedded within the chip and actuated by conventional means, or may be external to the chip, “squeezing” the respective passage closed by relying on the elastomeric nature of the device. Devices of rigid construction must rely on internal valving, whether internally actuated or externally actuated, for example by electromagnetic fields. Suitable valves are known to those skilled in the art. Toggling of valves from one reservoir to the next following fertilization can be used to simulate fluctuating nutrient content in vitro systems.

The reservoirs and microchannels of a gravity pump device is shown in FIG. 5. In this device, the supply reservoir is considerably greater in volume than the microchannel volume so that flow may occur for some length of time. In FIG. 5, the supply reservoir 68 is somewhat larger in area and taller than the outlet reservoir 69. When filled with fluid, the height difference of the two reservoirs allows fluid flow through microchannel 70 until the heights of fluid in the two reservoirs is equal.

The entire device may be micromachined from glass, metal, or other substrates, e.g. silica, but is preferably cast from thermoplastic or thermoset polymer using a “master.” Cast polydinethylsilicone elastomer is preferably used. Greater details of the preparation are discussed later. The sperm receiving reservoir and media reservoirs may be constructed with microtubing protruding into the respective reservoirs to supply fluid, or may communicate with a simple supply passage which exits the surface of the device and is suitably configured to receive the tip of a pipet, a syringe needle, etc., to supply fluid to the device. Likewise, the motile-sperm depleted fluid reservoir and sorted sperm reservoir, while not requiring a fluid supply, must ordinarily be at least vented in order that an airlock preventing fluid flow cannot be formed. The devices are preferably supplied fluid full, only requiring insertion of oocyte(s) and sperm sample into their respective locations, and initiation of pumping action to begin fluid flow.

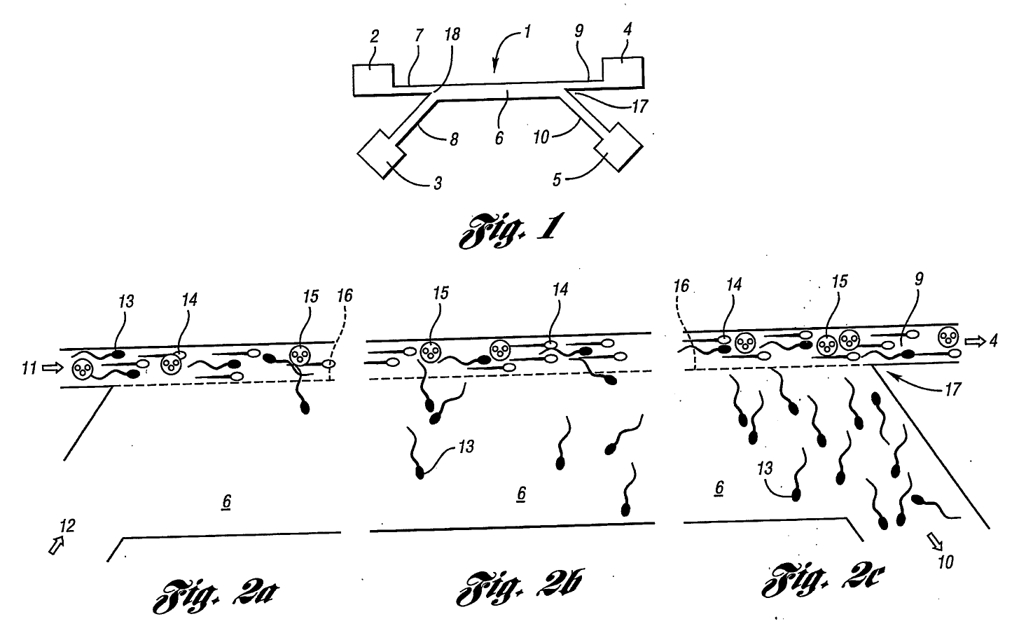

An understanding of the sperm sorting portion of the invention may be facilitated by reference to FIG. 1 which represents one relatively simple embodiment of an apparatus which may be used for practicing this function and by reference to FIG. 2 which illustrates sorting of motile from non-motile particles.

The device of FIG. 1 is shown in schematic form in plan, i.e. as viewed from above. The device 1 embodiment consists of a motile particle sort stream inlet 2 (or motile particle supply reservoir serving as an inlet), a media stream inlet 3 (or media reservoir), a motile particle-depleted sort stream outlet (or reservoir) 4, and a motile particle-enriched stream outlet (or reservoir) 5. Between the inlets 2 and 3 and the outlets 4 and 5 is located a sort channel 6. Connecting the sort channel 6 to the respective inlets and outlets are sort stream inlet channel 7, media stream inlet channel 8, motile particle-depleted sort stream outlet channel 9, and motile particle-enriched media stream outlet channel 10. The width of the sort stream channel must be large enough to allow the particles of interest to pass through effectively without blockage, as is also true of both outlet streams. In general, the inlet streams and outlet streams will have a cross-sectional area less than the sort channel. The relative cross-sections will be dependent on the flow volume and flow rates of the various streams. The linear flow rates are preferably similar, although the relative flow rates are only limited by the occurrence of mixing between the sort stream and the media stream. Depending upon numerous factors such as the viscosities of the media and sort streams, the motility of the particles, and the presence or absence of particles or debris of different size than the particles desired to be sorted, the volume of the media stream may be less than, substantially the same as, or greater than the volume of the sort stream over any section of the sort channel.

In operation, a supply of sperm is introduced into sort stream inlet 2 and caused to flow toward motile particle-depleted sort stream outlet 9, initially through channel 7, then through sort channel 6, and next to outlet channel 9. A media supply stream compatible (i.e. not destructive) of the sperm to be sorted is introduced into media stream inlet 3 and caused to flow through channel 8 into sort channel 6, through channel 10, and into motile particle-enriched media outlet 5. At the confluence of channels 7 and 8, a non-mixing, and preferably laminar flow is created, such that the sort stream and media stream flow in parallel through the sort channel. Non-motile (or lesser-motile) particles tend to remain in the sort stream, while motile particles move about randomly and enter the media stream. As a result of this random movement, the sort stream becomes depleted of motile sperm, while the media stream becomes increasingly enriched.

The invention may further be described broadly with reference to FIGS. 2a, 2b, and 2c, which illustrate pictorially the separation of motile from non-motile sperm and other non-motile particles in the sort channel of the device of FIG. 1. In FIG. 2a, the sort channel 6 is shown, beginning at the point of confluence of the sort stream 11 and the media stream 12. The sort stream 11 contains motile 13 and non-motile sperm 14 as well as other non-motile particles, here designated as “round cells” 15. Note that the size of the media stream in plan, and hence its volume, is considerably greater than the sort stream. Since sperm (and similarly, other motile particles) assume an essentially random distribution in the total liquid within a short period, a larger media stream volume will necessarily contain a larger fraction of total motile sperm 13.

In FIG. 2b, the randomization of motile sperm between the two streams has begun, and continues until the desired degree of randomization has been achieved. This degree of randomization is preferably such that the concentration of motile sperm in the media phase per unit of volume is the same or greater than the concentration per unit volume in the sort stream. Note that the sort stream and media stream are maintained as separate streams, each exhibiting laminar flow, and having a common boundary, or interface, 16. Greater concentration of motile particles in the media stream over the amount dictated by pure randomization may be achievable by employing a media stream in which the motile particles have increased mobility, i.e. by selecting a media stream less viscous than the sort stream, or by including additives which increase mobility of motile sperm relative to non-motile or poorly motile sperm.

In FIG. 2c, the motile sperm-enriched media stream is harvested by diverting it to flow into the motile particle-enriched channel 10, while the now motile particle-depleted sort stream continues through channel 9 into outlet 4.

The diverting juncture 17 which separates the motile particle-depleted sort stream from the motile particle-enriched stream may be of any geometry which avoids substantial mixing of the streams at this point. The juncture 17 may be positioned, for example, to provide for substantially the same outlet channel configuration (i.e. height, width) of the sort stream inlet, at this point. To minimize contamination of the media stream by non-motile sperm, the juncture 17 may also be configured such that a small portion of the media stream is also directed to the motile particle-depleted sort stream outlet 9. In this case, a modest loss of motile sperm will occur, however, the probability that non-motile sperm may enter the motile sperm-enriched media stream will be lessened as a result.

The nature of the sort stream is not critical. The sort stream may be a biologically derivel stream such as semen, or may be washed, diluted, maybe treated with additives, stains or fluorescing dyes, viscosity modifiers, may be buffered, etc., so long as the treatment does not impair the viability of the desired exit stream (motile particle-depleted or motile particle-enriched).

The media stream may be selected with the same considerations in mind which are applied to selection or modification of the sort stream. In some cases, the media stream may be water, but for biological systems, it is typical to employ fluids which maintain or enhance biological activity, such as physiological saline, buffered saline, nutrient broths, and the like. In the case of human sperm, the preferred media is HEPES buffered human tubal fluid.

The nature of the media fluid and the sort fluid may be selected, if possible, to avoid interfacial mixing due to osmotic effects. This is the case, for example, when the base fluid (e.g. water) of both the sort and media streams have substantially the same amounts of soluble ingredients such as salts, acids, bases, buffers, dissolved organic material, and the like. The fluids may also be selected, when possible, to avoid interfacial mixing by diffusion. However, complete absence of any diffusion is an unlikely goal in this respect.

The relative fluid volumes may be selected with respect to the desired degree of incorporation of the motile particles within the media phase. For the highest degree of incorporation, the media volume should be large with respect to the sort fluid volume. However, proportionately smaller media volumes may also be used, particularly when sequential (serial) sorting is performed. Ratios of media fluid to sort fluid of from 1:100 to 100:1, preferably within the range of 1:10 to 10:1 may be used. For typical applications, the ratio of media volume to sort fluid volume is within the range of 1:1 to 3:1. The media fluid volume is most preferably higher than the sort fluid volume.

The linear flow rates of the sort fluid and media fluid are preferably substantially the same, i.e. within a range of flow rates of 1.5:1 to 1:1.5. If the linear flow rate of the media fluid is greater than that of the sort fluid, correspondingly less transverse volume of media fluid can be used for the same degree of motile particle incorporation. Flow is preferably concurrent, although counterconcurrent flow is also possible provided that interfacial mixing is not exacerbated beyond that which facilitates the desired degree of depletion/enrichment of the sort and media fluids.

The interface between the sort and media fluids is preferably a substantially non-mixing interface. By “non-mixing” is meant an absence of mixing which occurs due to excessive turbulence between the two fluids. For example, it is most desired that parallel, concurrent, laminar flow take place such that a substantially “static” appearing interface is obtained, as opposed to an interface which exhibits waves, currents, eddys, and the like. Turbulent flow generally results in partial to full mixing of the streams, rendering depletion/enrichment of motile particles less efficient or even completely impossible. The theoretically best resolution of motile particles occurs when a static-appearing interface or “streamline” is created where interfacial mixing occurs only due to diffusional and osmotic effects. However, it would not depart from the spirit of the invention to allow some turbulence along the interface. The turbulence is excessive when the desired degree of resolution cannot be obtained, even with multiple stages of devices. The turbulence, expressed as a Reynolds number, should in any case be less than 2000, more preferably less than 100, yet more preferably less than 10, and most preferably 1 or less. High performance devices such as those illustrated by example herein, exhibit a Reynolds number of approximately 0.1.

The nature of the interface, i.e. its degree of turbulence, may be assessed by the degree of resolution. However, the turbulence may also be assessed in numerous additional ways. For example, in PDMS devices as described hereinafter, the optically transparent nature of the device allows the interface itself to be observed microscopically, for example by coloring one or both of the fluids and observing the interface by the change of color at the interface. By conventional optical techniques, the interface between media of differing refractive index are also easily observed. The degree of mixing of the sort and media streams may also be monitored by introducing a taggant, i.e. a radioactive soluble compound or non-motile particle, a visual or fluorescent dye, etc., into one stream but not the other. Appearance of the taggant in the outlet stream of the stream initially containing no taggant provides evidence of interfacial mixing, either of a turbulent kind, or by diffusion or osmosis. Some incorporation due to the latter two effects is expected, but is also expected to be quite minimal. An incorporation of 50% of the taggant into the non-tagged stream essentially constitutes complete mixing. Mixing of less than 20% of the taggant into the non-tagged stream, preferably less than 10%, more preferably less than 5%, and most preferably less than 1% is desired. So long as the Reynolds number is kept reasonably low, the degree of turbulence will be satisfactory. A flow which satisfies the above criteria is termed a “substantially non-turbulent flow” herein. It should be noted that concurrent flow streams exhibit much less turbulence, and hence interfacial mixing, than countercurrent flow streams.

Provided the fluid flow rate meets the non-turbulent requirements just described, the rate itself may vary widely. The walls of the sorting device also create the possibility for turbulence, since they are static with respect to the fluid flow. The effect of the walls will be most important when narrow channels are employed, and particularly at the walls which abut the narrower of the sort or media streams. Since the devices of interest are rather small and have rather small channels, linear flow rates of less than 10 cm/s, preferably less than 10 mm/s are preferred. Flow rates of between 0.1 mm/s to 10 mm/s are particularly preferred. The low end of linear flow rate is determined by the mixing of non-motile particles from the sort stream into the media stream by Brownian motion. For example, at a flow rate of zero, with identical base fluid compositions (e.g. buffered saline), distribution of non-motile particles into the media phase would eventually be complete over time such that their concentrations become identical. The higher the flow rate, the less Brownian redistribution of non-motile particles will occur. The upper limit of the flow rate is reached when the interfacial flow becomes turbulent; as evidenced by a high degree of mixing.

Determining the relative flow volumes, relative flow rates, and absolute flow rates of any given stream can be routinely accomplished by one skilled in the art by simple calculations and/or measurements of resolution, for example by varying the respective rates and volumes and determining the relative enrichment and depletion of particles between the sort and media streams.

The geometry of the devices can vary. Sort channel length, for example, is generally a function of the rapidity at which motile particles randomize themselves between the two phases, and the flow rates. For example, at a given flow rate, motile particles which have limited motility will require a longer sort channel, while at a given sort channel length, less motile particles will require a slower rate of flow. Interfacial surface area also effects the geometry of the device. For example, flat rectangular sort channels with one fluid located parallel to and abutting a channel face of greater dimension, and with the other fluid adjacent, will exhibit faster randomization and thus require less sort channel length than the same channel when the first fluid is located parallel to and abutting a channel face of lesser dimension. In the latter case, the interfacial area is much reduced as compared to the former. For most purposes, the sort channel will be quite short, in. almost all cases less than 2-5 cm, and for most devices, in the range of 100 μm to 1 cm. For sperm sorting, for example, a sort channel length of 5000 μm (5 mm) has proven quite satisfactory. In staged devices, shorter sort channel lengths may be desirable.

The cross-sectional geometry of the sort channel is not critical. Square, rectangular, ellipsoidal, circular, trapezoidal, triangular or other cross-sections may be used. For ease of manufacturing, non-undercut channels such as square, rectangular, triangular, trapezoidal, and half-round or half-elliptical sections are preferred. These shapes are preferred, for example, when neat casting or solution casting methods of construction are employed. In the case of construction by stereolithography techniques (“SLA”), more complex shapes can easily be fabricated. Complex shapes with undercut channels can also be provided by casting techniques when the device is cast in successive layers which are then attached together, for example by bonding. However, the channel width must be such that both the media stream and sort stream can both incorporate particles. For human sperm sorting, for example, a substantially rectangular channel with a height of 50 μm and a width of 500 μm has proven satisfactory. For a point of reference, human sperm have a head of about 2.5 μm in diameter and about 5 μm long, and are about 60 μm in overall length.

The cross-sectional areas and hence size of the supply channels and outlet channels are generally smaller than those of the sort channel. The minimum size of the sort stream inlet channel is dictated by the size of the particles which are present in the sort stream. Preferably, the sort stream channel provides a free channel from 3 to 10 times the size of the particles expected to be contained therein. The same considerations apply to the size of the media stream outlet channel, but not necessarily to the media stream inlet channel. Preferably, the sort stream inlet and outlet channels will have comparable sizes, although in some instances, as described earlier, it may be desirable that the outlet channel is larger than the inlet, thus incorporating a portion of the media stream into the sort stream. For sperm sorting, a rectangular sort stream inlet channel of 50 μm height, 100 μm width, and 5000 μm length has proven satisfactory.

The length of the various inlet and outlet channels is not critical. It is preferred that at least the inlet channels have some substantial length, to encourage formation of a laminar flow stream prior to the confluence of the sort and media stream channels. In general, more viscous fluids will not require as long a channel length as less viscous fluids. In some cases, the inlet channels may be completely dispensed with, i.e. the sort stream inlet (or reservoir) and/or media stream inlet (or reservoir) may feed directly into the sort channel. For most purposes, however, and to facilitate construction of sorting devices, it is preferable that inlet channels be employed. For the sperm sorting device described later, for example, inlet channel lengths of about 3 mm have proven satisfactory.

The junction 18 (FIG. 1) of confluence of the sort and media streams is preferably configured to encourage a smooth joining of the fluid streams without excessive mixing. In general, therefore, the junction will be a relatively acute angle. The included angles between the sort stream inlet channel and the sort channel and between the media stream inlet channel and sorts channel may be the same or different, i.e. the devices are not necessarily symmetrical. The same considerations apply to the junction 17 where the sort stream and media stream are separated, or “diverted” from each other. However, it is preferred that the sort stream inlet channel, sort channel, and sort stream outlet channel be substantially linear to provide as little disturbance of the non-motile particles in the sort stream as possible.

The material of construction of the devices may be any suitable material, and the fabrication of the device may involve any fabrication process. For example, devices may be micromachined chemically by etching of glass, silica, silicon, metals, or by solution etching of polymers, etc. The devices may also be individually fabricated by known stereolithography techniques. The devices may be injection molded of moldable polymers, for example silicone rubber, thermoplastic polyurethane, polyethylene, polypropylene, polytetrafluoroethylene, polyvinyl chloride, polyvinylidene chloride, polyamide, polyester, and the like.

It is at present preferable to cast the devices by supplying a negative “master” and casting a castable material over the master. Preferred castable materials are polymers, including epoxy resins, curable polyurethane elastomers, polymer solutions, i.e. solutions of acrylate polymers in methylene chloride or other solvents, and preferably, curable polyorganosiloxanes, most preferably for cost reasons, polyorganosiloxanes which predominately bear methyl groups, such as polydimethylsiloxanes (“PDMS”). Curable PDMS polymers are well known and available from many sources. Both addition curable and condensation-curable systems are available, as also are peroxide-cured systems. All these PDMS polymers have a small proportion of reactive groups which react to form crosslinks and/or cause chain extension during cure. Both one part (RTV-1) and two part (RTV-2) systems are available. Addition curable systems are preferred when biological particle viability is essential.

In many instances, transparent devices are desirable. Such devices may be made of glass or transparent polymers. PDMS polymers are well suited for transparent devices. A benefit of employing a polymer which is slightly elastomeric is the case of removal from the mold and the potential for providing undercut channels, which is generally not possible with hard, rigid materials. Methods of fabrication of microfluidic devices by casting of silicone polymers is well known. See, e.g. D. C. Duffy et al., “Rapid Prototyping of Microfluidic Systems in Poly(dimethylsiloxane),” ANALYNICAL CHEMISTRY 70, 4974-4984 (1998). See also, J. R. Anderson et al., ANALYTICAL CHEMISTRY 72, 3158-64 (2000); and M. A. Unger et al., SCIENCE 288, 113-16 (2000); J. C. McDonald, et al, “Poly(dimethylsiloxane) as a material for fabricating microfluidic devices,” ACCOUNTS OF CHEMICAL RESEARCH, 35, 491-99. For devices of polymethylmethacrylate, see L. Martynova, et al, “Fabrication of Plastic Microfluidic Channels by Imprinting Methods,” ANALYTICAL CHEMISTRY 69, 4783-4789.

The nature of the channel and reservoir walls of the devices may be selected in view of the application of the device and the fluids contemplated for use therein. In addition, the walls may be treated or derivitized to modify their surfaces with biologically compatible or bioactive coatings, or to provide chemical functionality. For sperm sorting, coating the channels with bovine serum albumin (BSA) has proven useful in improving liquid flow within the channels and to minimize non-specific adsorption of cells to channel walls.

The oocyte insemination chamber and the sorted sperm receiving reservoir may be a single reservoir performing both functions. Alternatively, a separate insemination chamber may be provided. In the latter case, the insemination chamber and sorted sperm reservoir are preferably connected by a flow channel which will allow sperm-containing liquid to flow into the insemination chamber. This flow may be valved, and initiated by gravity pumping, as with the media reservoir and sperm reservoir, or may require assist in the nature of pressure or by vacuum, i.e. aspiration, to initiate or cause (maintain) flow.

The insemination chamber may be sized in accordance to the number of oocytes desired to be introduced, and their species-dependent size. The chamber must have a means for introducing one or more oocytes into the chamber. The chamber may be configured with narrowings to condition the oocytes, when desired, i.e. to remove cumulus cells or to force them to the poles of the oocyte. Techniques for micromanipulation of oocytes are known. See, e.g. Beebe et al., “Microfluidic Technology for Assisted Reproduction,” THERIOGENOLOGY, 57 125-135 (2002). For example, the chamber may communicate with the outside of the device with a funnel shaped opening configured to receive a pipet tip, and having an “oocyte duct” which communicates with the insemination chamber and which is of sufficient diameter so as to allow oocytes to pass.

While the insemination chamber may be a static volume, it is much preferred to establish fluid flow through the chamber in order to continue to supply new sperm sources, nutrient media, and the like, and to remove products of cellular metabolism. Thus, the oocyte chamber must, in general, contain a barrier which contains holes or passages which permit the flow of fluids and sperm, but which are sized so as not to allow the oocytes to travel through the barrier. The barrier may in principle be a wall of the insemination chamber having a small exit hole or channel for fluid and sperm to pass. However, it is preferably to provide for further holes or channels which will serve to appreciably prevent stagnant volumes where metabolic byproducts and/or cellular toxins may accumulate. It must be kept in mind that microfluidics devices are quite small, and thus barrier structures which may be contemplated for macroscopic devices may be incapable of fabrication in microdevices.

One barrier structure 46 which has been found to work well is illustrated in FIG. 4. Like many microfluidics devices, this barrier design is advantageously created from multiple layers of cast polymer, which are then assembled into the finished device. In FIG. 4, a lower layer 60 is configured to contain a blocking wall 61 of a height typical of an oocyte 62 to be deposited in the chamber, preferably somewhat taller, and of a thickness 63. An upper layer 64 is configured to contain multiple parallel grooves or channels 65 which are longer than the thickness 63, such that when the second layer is affixed to the first layer with the channels directly over the blocking wall, a multiplicity of passages over the blocking wall are created. These channels may extend transversely over the entire width of the insemination chamber. Such a device permits virtually unimpeded flow while still serving as a barrier to the oocyte.

A simple device for microfluidic insemination consists of a microchannel sized to receive one or more oocytes, and containing a barrier as described with the channel. A funnel-shaped or other shaped opening allows injection or pipetting of oocytes into the channel, and continued liquid flow causes the oocytes to travel down the channel to the barrier. The barrier and microchannel thus together comprise an insemination chamber. Sperm containing liquid may then be allowed to flow into and preferably through the chamber. Following fertilization, the liquid flow may be replaced by nutrient-containing liquid. Various liquids may be alternated to simulate in vivo systems.

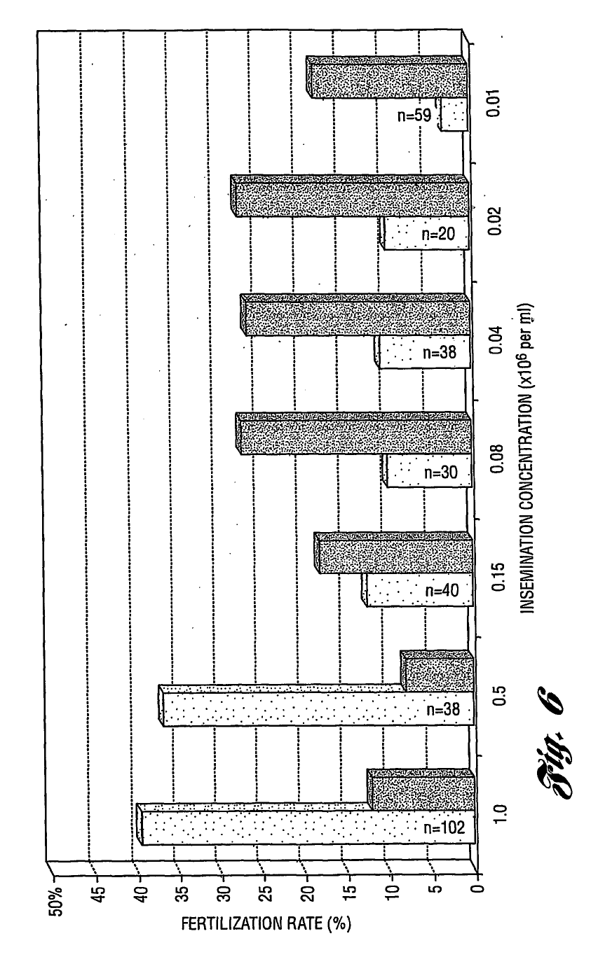

FIG. 3 illustrates one embodiment of an integral sperm sorting and insemination device 30. The drawing depicts the passages in the device, which is preferably constructed by assembling multiple layers of cast silicone elastomer. The sort channel 31 is supplied with sperm from sperm receiving reservoir 32, which has a passage 33 leading to the exterior of the PDMS device, and configured with a funnel like opening 34 so as to more readily receive a pipet tip or other delivery device. The reservoir 32 has a height and width such that as fluid flows outwards into channel 31, the fluid level will not drop, but instead, a “plug” of fluid will move horizontally. Sperm-containing liquid, e.g. semen, is introduced into the reservoir through passage 33.

Sort media fluid is introduced into sort media reservoir 35 through passage 36, terminated by a funnel shaped opening 37. From the sort media reservoir, fluid flows through media channel 38 to sort channel 31. At the confluence of the sperm liquid stream and the sort media stream, sort channel 31 widens to accommodate the co-laminar streams, and this widened portion 39 continues up to the point of separation 40 of the streams into a continuing stream 41 which flows into a depleted sperm reservoir 42, preferably having an outlet or vent 43 to the outside. The sort media, now enriched with more motile sperm, flows through sorted sperm channel 44 to oocyte chamber 45. The oocyte chamber 45 may be of the same dimensions as channel 44, or may, as shown here, be larger. Prior to and following the oocyte insemination chamber 45 are located barriers 46 which allow media fluid and sperm to pass, but which do not allow an oocyte to pass. The barrier may be similar to that of FIG. 4, for example. From the chamber 45, the fluid flow continues to the sorted sperm reservoir 48, which may also have an outlet or vent 49.

Delivery of one or more oocytes into chamber 45 may be accomplished via oocyte duct 50, which is terminated by a funnel shaped opening 51. The interior dimensions of duct 50 are such that oocytes may pass through the duct into chamber 45. A portion 52 of duct 50 is horizontal prior to entry into the chamber 45, to allow for valving by valve 53. Valve 53 may be an internal, actively initiated valve, or may be an external valve, for example a electromagnetically or piezoelectrically actuated tactile sensor as might be found on a programmable braile display. By valving the oocyte duct closed following oocyte placement, the height of the oocyte duct will not affect the fluid flow through the device. Following fertilization, the oocyte may be removed by disassembling the device, or by opening valve 53 and withdrawing the oocyte by suction back through oocyte duct 50.

The device of FIG. 3 is a “pass through” inseminator, since the sorted sperm flow by the oocyte on their way to the sorted sperm reservoir. The valved oocyte duct may be connected directly to sorted sperm reservoir 48 to provide an insemination chamber or well rather than a channel. Note the outlet has been provided with a simple “pin” barrier 47 to prevent egress of the egg from the chamber. Another device is shown in FIG. 7, wherein greater flexibility in oocyte/sperm contact is possible. Equivalent portions of FIG. 7 are labeled similarly to FIG. 3. However, in FIG. 7, sorted sperm flow directly to sorted sperm reservoir 48. From reservoir 48, a sperm channel 55 directs sperm to oocyte chamber 45, from which oocytes are prevented from leaving by barrier grates 46. Oocyte(s) are delivered into chamber 45 by oocyte duct 50.

The sperm channel 55 is configured so as to be valved between the sorted sperm reservoir 48 and oocyte chamber 45, e.g. by a “braille” actuator valve 56. Moreover, on the side of sperm channel 55 remote from sorted sperm reservoir, an active or passive pump may be positioned, in this case, a series of three or more braile actuators 56 which press on the channel from outside in a sequential rhythmic pattern, which causes a peristaltic-like pumping action. When valve 56 is open and the pump 57 is energized, media fluid containing sorted sperm will be pumped from the sorted sperm reservoir 48 to the oocyte insemination chamber 45, and from there to a receiving reservoir 58.

Within microfluidic culture environments for human and non-human embryos, a dynamic culture system holds numerous advantages in comparison to current static culture systems. First, gradual movement of media over embryos would have the ability to remove metabolic by-products such as ammonia and oxygen free radicals which are detrimental to embryo development. In addition, individual blastomeres (cells) that comprise the embryo can undergo apoptotic death, fragmentation and release of apoptotic cell death agents that may be detrimental to survival of remaining blastomeres. A dynamic culture system would remove such agents. Second, current human embryo culture strategies use 2-3 sequential media for 3-6 day culture with abrupt media changes, which can inflict osmotic stress upon embryos. A dynamic culture system would allow gradual media changes that may be beneficial. Third, within the oviduct, cilia of epithelial cells are continuously “beating” causing constant movement of preimplantation embryo(s). Such movement, which can be achieved with dynamic media flow, may be beneficial for establishing poles-of-cell division and enhance embryo developmental competence. Fourth, dynamic media flow over embryos will allow “sampling” of embryo by-products that have potential of indicating which embryos have the greatest change of implantation and pregnancy establishment. Lastly, group embryo culture is believed to be superior to individual culture based on the idea that more developmentally advanced embryos (“helpers”) produce substances that enhance poorer embryo (“lagger”) development. Conversely, poorer developing embryos may have detrimental influence on more advanced embryos. Culture devices using dynamic media flow may facilitate “helper” embryo influences on “lagger” embryo development without negative reciprocal effects. For the effects of fluid flow on embryo development, reference may be had to S. Nonaka et al., Determination of Left-Right Patterning of the Mouse Embryo by Artificial Nodal Flow,” NATURE, 418, pp. 96-99, July, 2002.

It has been surprisingly discovered that the microfluidics device described above is more efficient than conventional methods of fertilization when sperm concentration is low. For example, with B6C3F1 mice oocytes, the recommended sperm concentration is 1×10⁶ sperm/mL. When this concentration is used in a microfluidics device, the fertilization efficience is considerably lower, about 12% (% of oocytes fertilized) as opposed to about 37% in standard center-well equipment. The fertilization rate at a lower concentration of 0.5×10⁶ sperm/mL fared even worse in the microfluidics device, at about 8% versus about 36% for the center-well IVF.

However, when the concentration was lowered to below 0.5×10⁶ sperm/mL, significant and surprising changes occurred. At 0.15×10⁶ sperm/mL, the microfluidics chamber exhibited an 18% success rate, whereas center-well IVF was about 12%. At concentrations of 0.08×10⁶ to 0.02×10⁶ sperm/mL, the microfluidics IVF was more then twice as efficient. These results are shown in FIG. 6. Thus, the subject invention also pertains to a method of increasing fertilization rate at low sperm concentrations, by employing a device of the subject invention. The improvement is assessed by comparing the average rate of fertilization in the microfluidics device to the rate of fertilization in a center-well technique, using the same sperm concentration, same media, and same temperature. The improvement may be assessed employing sperm and oocytes from a single species, or the results obtained from different species may be averaged. If statistical data for a given species, i.e. human, bovine, cannot be obtained due to difficulties in collecting suitable data or to expense, it may be assumed, for the purpose of assessing the scope of the claims, that the mouse species, in particular the B6C3F1 variant, will determine the improvement.

Having generally described this invention, a further understanding can be obtained by reference to certain specific examples which are provided herein for purposes of illustration only and are not intended to be limiting unless otherwise specified.

### EXAMPLES

### Example 1

A microfluidic sperm sorting device is prepared from Dow Corning SYLGARD® 184 curable silicone resin, using the soft lithography technique described by D. C. Duffy et al., cited previously. The curable PDMS is cast onto a master having the desired reservoir and channel features as protuberances. The cast PDMS sorting devices are plasma oxidized to seal the open channel side of the casting to a glass cover slide. Channels and reservoirs are coated with 1% bovine serum albumin fraction V from Sigma, dissolved in phosphate buffered saline (PBS) from Invitrogen Corporation. The entire device is approximately 6 mm thick, exclusive of the cover slide, and somewhat larger than a U.S. penny coin. A perspective view of the device is shown in FIG. 1.

In FIG. 1, the PDMS casting is transparent, and only the reservoirs and channels are depicted. A glass cover slide or other substrate would be bonded to the bottom plane of the device. The channels are rectangular in cross-section, with a channel height of 50 μm, while the reservoirs are roughly semi-circular. Both inlet reservoirs 2 and 3 are approximately 3 mm in height, while the outlet reservoirs are approximately 2 mm in height. The inlet and outlet channels 7, 8, 9, 10 are about 5000 μm long. The sperm inlet channel 7 and the motile depleted sperm outlet channel 9 have a width of 100 μm, while the media inlet channel 8 and outlet channel 10 have a width of about 300 μm. The sort channel 6 has a width of 500 μm and a length of 5000 μm.

Semen samples were obtained with institution Review Board approval from men undergoing infertility evaluation. Sorting tests were performed using washed semen samples. In the order listed, 60 μL of processing media was added to the media inlet reservoir, 50 μL of a washed semen sample to the sample inlet reservoir, and 2 μL of media to each of the outlet reservoirs. Sperm sorting yields were calculated taking these dilution factors into account. The numbers of motile sperm were determined by a Makler Counting Chamber (Sefi-Medical Instruments, Haifi, Israel). For visualization of membrane-compromised sperm, which generally corresponds to non-motile sperm, 3 μL of propidium iodide (Molecular Probes, www.probes.com, 60 mM dissolved in processing media) was added to sperm samples prior to sorting. A Texas Red filter set (577 nm excitation, 620 nm emission) was used to view red fluorescence from stained cells. An inverted microscope (NIKON TE 300, www.nikon-usa.com) with a CCD camera (Hamamatsu ORCA-100, www.hamamatsu.com) was used to capture images and record movies.

The sorting device uses a sorting system where non-motile sperm flow along their initial streamlines and exit one outlet whereas motile sperm can deviate from their initial streamlines and exit through a different outlet. This sorting mechanism is related to the “filtering” mechanism used in an “H-filter” where rapidly diffusing small molecules exit through a different outlet from larger molecules and particles that diffuse more slowly. The difference between the two devices is that the sorting device of the present invention takes advantage of active movement of cells whereas an H-filter takes advantage of passive diffusion of particles. This type of sorting is possible because in small channels, multiple laminar streams can flow parallel to each other with no turbulent mixing at the interface between the streams. Typical Reynolds Numbers for the flow of sperm sample and media inside the sorting device were on the order of 0.1. Non-motile human sperm, approximately 60 μm in length, and non-motile particles on the same order of magnitude in size diffuse slowly (D=1.5×10⁻¹³ m²/sec; 690 sec to diffuse 10 μm) and remained within their initial streamlines. In contrast, motile human sperm swim at velocities greater than 20 μm/sec at 20° C. This rapid mobility allows motile sperm, but not the non-motile sperm, to distribute themselves randomly within the width of a 500 μm channel within seconds. The sorting device was designed specifically to give sperm a residence time of 20 seconds in the main separation channel. A bifurcation placed at the end of this separation channel allows efficient collection of only the motile sperm that deviated from its initial inlet stream.

The sorting device described integrates all functions necessary for sperm sorting, for example, inlet/outlet ports, fluid reservoirs, pumps, power source, sort channel, etc., onto a simple chip design that is practical to manufacture and use. A key design feature of this embodiment is the set of four horizontally-oriented fluid reservoirs that also function as sample inlet/outlet ports and a fluid pumping system. The orientation, geometry, and size of these reservoirs are designed to balance gravitational forces and surface tension forces, and provide a pumping system that generates a steady flow rate over extended periods of time regardless of the volume of fluid in the reservoirs. This contrasts with conventional gravity-driven pumping systems whose flow rates decrease over time as the volume of fluid in the inlet reservoir decreases. The diameters of the reservoirs were selected to be small enough that surface tension prevents liquid from spilling out of the horizontally-oriented reservoirs, but large enough to hold sufficient amounts of sample (tens to hundreds of microliters) and allow convenient sample introduction and recovery. This balance of forces allows the reservoirs to be arranged horizontally without the liquid inside spilling out. The horizontal reservoir arrangement, in turn, holds the height difference between the fluid in the inlet and outlet reservoirs the same (1.0 mm height difference between inlet and outlet reservoir ceilings) regardless of the volume of fluid present in the reservoirs and maintains a constant hydraulic pressure even as the amount of fluid in the reservoirs changes.

The passively-driven pumping system described here is unique in that it uses horizontally-oriented reservoirs to overcome the problem of traditional gravity-driven pumping, where the pressure decreases as the amount of liquid in the reservoir decreases. Furthermore, the structure of the pump is greatly simplified compared to other mechanical or non-mechanical pumping systems allowing easy manufacture and integration of the pump into a small, integrated device. Finally, the use of gravity and surface tension as the driving-force contributes to the overall small size of the sorting device by eliminating the need for power supplies such as batteries. Taking gravity, surface tension, and channel resistance into consideration, the sorting device was designed to give a steady flow- rate of sperm with a residence time of approximately 20 seconds inside the main sort channel. More specifically, the device is designed so that the flow resistance of the fluid reservoirs is more than 10 times less than that of the microfluidic channels, and therefore negligible. Thus, the resistance of the channels, calculated to be 2.8×10¹² kg/(sec/m⁴), approximates the total resistance of the system. Since a bulk flow rate of 0.008 μL/sec is required to achieve the desired residence time of 20 seconds and the total resistance is 2.8×10¹² kg/(sec/m⁴), the net pressure drop required to drive the fluid is 23 N/m². To achieve this desired pressure drop, we designed the dimensions of the reservoirs such that capillary forces (3.0 mm diameter inlet reservoir vs. 2.0 mm diameter outlet reservoir) would be 13 N/m² and the pressure drop across the microfluidic channel of the sorting device due to hydrostatic forces (1.0 mm height difference) would be 9.8 N/m². For calculation of the capillary force, the contact angle was assumed to be 0° (the contact angle of water on BSA coated PDMS is very small), the surface tension of the washed semen sample assumed to be approximately 0.040 N/m (less than that of water due to “impurities” such as proteins), and the viscosity of the washed semen sample to be similar to that of water. The observed bulk flow rate of 0.008 μL/sec for a dilute particle suspension in 1% BSA solution was approximately equal to that of the calculated flow rate. Actual sperm samples sometimes had lower flow rates due to larger apparent viscosity. Smaller flow rates for the sperm sample stream would result in slightly lower yields but does not affect the purity of the sperm recovered at the sorted sperm outlet.

Sperm sorting efficiencies of the sorting device were evaluated by three methods: (i) tracking the movement of motile sperm in the channel by phase contrast microscopy, (ii) tracking movement of propidium iodide (PI) stained cells in the channel by fluorescence microscopy, (iii) using a Makler Counting Chamber, a grid-based sperm counting device, to determine numbers of motile sperm and non-motile sperm in the inlet and outlet reservoirs (FIG. 8). The sperm tracking experiments shows the process of how motile sperm can swim out of its initial streamnline. PI stains membrane compromised cells such as dead cells, and thus allows the non-motile sperm to be highlighted and visualized with red fluorescence while the motile sperm remain unstained. The bar graphs in FIG. 8 compare percentage of sperm that are motile before and after sorting. The unshaded bars represent the initial sperm sample, while the solid bars represent the motile particle-enriched media stream. The purity of motile sperm after sorting was nearly 100% regardless of motile sperm purity before sorting. The yields (39%, 42%, 43%), defined as the ratio of the number of motile sperm in the motile sperm outlet reservoir to the total number of motile sperm in the sperm sample inlet reservoir, were comparable to or greater than the recovery rates (0.8% to 50%) of sperm processed using conventional sorting methods such as direct swim-up, swim-up from a pellet of centrifuged sperm, or density gradient separation. It was also observed that sperm morphology, another important trait that correlates with successful pregnancies, also improved after sorting with the device (Strict Sperm Morphology: 9.5±1.1% normal before sorting to 22.4±3.3% normal after sorting). Kruger Strict sperm morphology is a set of criteria or standards whereby sperm must fit within specific measurements (head width and length, tail length, acrosome making up a certain percentage of the sperm head) and lack abnormalities (e.g. pin head, round head, crimped tail).

### Example 2

A device similar to that of Example 1 is configured with a funnel shaped oocyte duct communicating with the channel leading to the sorted sperm reservoir, as shown in FIG. 3. Prior to the beginning of sperm sorting, a mouse cumulus mass (20-30 oocytes) is introduced by means of a pipet. Mouse sperm is introduced into the sperm reservoir and media fluid into the media fluid (sort fluid) reservoir. One co-laminar flow begins, more motile sperm cross over the interface between the co-flowing streams, and the sperm-enriched media fluid flows into the sorted sperm reservoir collection well (volume: ca. 30-40 μL), which also serves as the insemination chamber. Successful fertilization is noted following 24 hours of co-incubation within the collection well (insemination chamber).

### Example 3

A microfluidic channel/insemination device is fabricated from PDMS by the layering technique. A single microchannel connects two larger reservoirs, one having a fuel shaped oocyte duct attached thereto, which also serves as a fluid inlet. The microchannel is 500 μm wide by 180 μm deep, and is about 2 cm long. After about 80% of its length, the channel contains a barrier grate as depicted by FIG. 4. The parallel channels of the barrier grate are prepared as a separate PDMS layer and subsequently bonded to the layer containing the channel.

### Example 4

Oocytes from B6C3F1 mice are introduced into the device of Example 3 by pipet, and buffered media is added to cause the oocytes to travel to the barrier grate where their further progress is blocked. Fertilization is achieved by adding sperm-containing media into the microchannel. Various concentrations of sperm are employed, and the fertilization rate compared to fertilization employing the same types of oocytes, media, and sperm, but by a center-well method. The results are given in FIG. 6.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. An integrated microfluidic sperm isolation and insemination device, comprising:
a) at least two gravity pump liquid reservoirs, one of said reservoir being a sperm receiving reservoir, one of said reservoirs being a sort media liquid reservoir;
b) at least one sort channel, said sort channel having a sort side and a reject side, and an upstream portion in fluid communication with said sperm receiving reservoir on said reject side of said sort channel, and in fluid communication with at least one sort media reservoir on said sort side of said sort channel;
c) a rejected sperm receiving reservoir in fluid communication with said downstream portion of said sort channel on said reject side of said sort channel;
d) a sorted sperm reservoir in fluid communication with said downstream portion of said sort channel on said sort side of said sort channel, and
e) an oocyte insemination chamber sized to contain one or more oocytes and into which sorted sperm flows, said chamber configured to contain at least one barrier which prevents egress of oocyte(s) located in said chamber but which is configured to allow fluid flow into or through said chamber,
wherein said oocyte insemination chamber and said sorted sperm reservoir may together be a single chamber, and wherein sperm-containing liquid in said sperm receiving reservoir and a sort media liquid in a second reservoir exhibit gravity induced co-laminar flow of both liquids through said sort channel in parallel but separate streams having an interface therebetween, wherein motile sperm cross said interface and are ultimately transported in said sort media liquid to said oocyte insemination chamber from said sort channel or from said sorted sperm reservoir.

2. The device of claim 1, constructed of a silicone elastomer.

3. The device of claim 1, having an oocyte duct communicating with said oocyte insemination chamber.

4. The device of claim 1, wherein said sorted sperm reservoir and said oocyte insemination chamber are a single chamber, also in communication with an oocyte duct.

5. The device of claim 1, wherein said barrier comprises a three dimensional barrier grate having forming plurality of barrier flow channels, said barrier flow channels smaller than an oocyte diameter by an amount such that said oocyte is blocked from passing through said barrier flow channels, but larger than the size of sperm such that sperm may travel through said barrier.

6. A method for in vitro insemination of an oocyte with sorted, motile sperm with minimal manipulation of said oocyte, said method comprising
a) selecting a device of claim 1;
b) introducing one or more oocytes into said oocyte insemination chamber;
c) introducing a first sperm-containing liquid to be sorted into a second sperm-containing liquid containing sperm of higher average motility than sperm of said first sperm containing liquid;
d) introducing a sort media liquid into said sort media liquid reservoir;
e) flowing said first sperm-containing liquid and said sort media liquid co-laminarly through said sort channel;
f) removing from said sort channel said second sperm-containing liquid; and
g) contacting said second sperm-containing liquid with said oocyte(s).

7. The method of claim 6, wherein said oocyte insemination chamber communicates with an oocyte duct, said method further comprising introducing said oocyte(s) into said oocyte insemination chamber through said oocyte duct, and removing one or more sperm-contacted oocytes from said oocyte insemination chamber through said oocyte duct.

8. The method of claim 7, wherein said sperm-contacted oocyte is a fertilized oocyte.

9. The process of claim 7, wherein introducing and removing of said oocyte(s) from said oocyte insemination chamber are performed with a pipet.

10. The method of claim 6,. wherein said oocyte insemination chamber is configured such that a plurality of oocytes occupying said chamber are forced to assume serial positions with respect to the direction of fluid flow.

11. The method of claim 6, wherein one or more oocytes are fertilized by sperm in said second sperm-containing liquid, and a growth media liquid is introduced into at least one of said gravity pump liquid reservoirs to provide a flow of growth media past said fertilized oocyte(s).

12. A method for improving the rate of fertilization of oocytes when employing low sperm concentration, comprising introducing one or more oocytes into a microfluidic channel, said channel having disposed therein a barrier having openings therein through which liquid and sperm may flow, but which are too small to allow an oocyte to enter;
introducing liquid and causing said oocyte(s) to travel through said channel to said barrier;
introducing a sperm-containing liquid of low sperm concentration into said channel and flowing said sperm-containing liquid past said oocyte(s), wherein the rate of fertilization at the sperm concentration used is higher than the rate achieved in center-well fertilization.

13. The method of claim 12, wherein the sperm concentration is less than 0.5×10⁶ sperm/mL.
